(19) United States
(12) Patent Application Publication
(10) Pub. No.: US 2012/0076795 A1
(43) Pub. Date: Mar. 29, 2012

(54) Anti-PECAM Therapy, Compositions, Methods, and Uses

(75) Inventor: Robert DEBS, San Francisco, CA (US)

(73) Assignee: Sutter West Bay Hospitals D/B/A California Pacific Medical Center, El Granada, CA (US)

(21) Appl. No.: 13/246,543

(22) Filed: Sep. 27, 2011

Related U.S. Application Data
(60) Provisional application No. 61/386,955, filed on Sep. 27, 2010.

(51) Int. Cl. A61K 39/395; A61P 35/00

(52) U.S. Cl. 424/152.1; 424/172.1

(57) ABSTRACT

The disclosure relates to the discovery that systemic administration of an antibody that binds to PECAM-1 increases body weight while suppressing the metastatic spread of a wide variety of different tumor types which are typically fatal in humans. This discovery provides a basis for the generation of novel treatments and medicaments, wherein provision of a systemic dosage of anti-pECAM-1 antibody or a proxy that provides the same functional result is administered to a patient suffering from cancer cachexia and/or metastasis associated with end-stage cancer

### I. CROSS REFERENCE TO RELATED APPLICATIONS

The application claims priority under 35 U.S.C. §119 to U.S. Provisional Application Ser. No. 61/386,955, filed 27 Sep. 2011, the disclosure of which is incorporated herein by reference.

### II. TECHNICAL FIELD

The disclosure relates to the identification of a method and related compositions for treating end-stage metastatic cancer and tumor-induced cachexia. The methods and compositions comprise a PECAM-binding agent, such as an antibody, and methods of treating or preventing end-stage metastatic cancer and tumor-induced cachexia using an PECAM-binding agent.

### III. BACKGROUND

Patients with advanced cancers become progressively more debilitated as tumors enter an accelerated stage of pre-terminal exponential growth. At this stage, patients develop a wasting syndrome known as cancer cachexia. Cancer cachexia (also referred to as tumor-induced cachexia) is characterized by progressive weight loss through the loss of skeletal muscle and adipose tissue. Cancer cachexia is common with many cancers especially with remission failures and advanced metastatic disease.

Loss of body weight in affected individuals could be about 10 to 20 percent. The degree of cachexia can be independent of the tumor size or the extent of metastatic disease. Cachexia reduces response to antineoplastic treatments and predisposes patients with advanced cancers to developing severe treatment-related toxicities. It produces death in more than 20 percent of patients.

Cachexia is a complex metabolic condition mediated by certain cytokines, and involves a decrease in protein synthesis as well as increased tissue catabolism. Tumor-induced cachexia can be due to anorexia, inflammation, metabolic alterations and/or enhanced muscle proteolysis.

Metastatic progression is characterized by exponential growth of tumors leading to multi-organ destruction culminating in a lethal pre-terminal stage. Antineoplastic treatment is typically stopped at this pre-terminal stage as existing treatments are ineffective. Furthermore, patients are too debilitated to tolerate any further antineoplastic treatments. They are prone to life-threatening complications from antineoplastic treatments that are well tolerated in patients with earlier stage cancers. Patients will subsequently be transitioned exclusively to palliative care with a focus on reducing the severity of the disease symptoms, to relieve suffering and to improve quality of life before imminent death.

Current treatments for cancer cachexia focus on relief of symptoms, rather than the underlying cancer. These treatments for cancer cachexia remain ineffective and do not effectively treat the pre-terminal stage of metastatic cancer. There exists a need in the art for a method and related compositions for treating end-stage metastatic cancer and cancer cachexia. The present disclosure fulfills this need and provides related aspects desired by practitioners in the field.

### IV. SUMMARY

The disclosure relates to the discovery that systemic administration of an antibody that binds to PECAM-1 increases body weight while suppressing the metastatic spread of a wide variety of different tumor types which are typically fatal in humans. This discovery provides a basis for the generation of novel treatments and medicaments, wherein provision of a systemic dosage of anti-pECAM-1 antibody or a proxy that provides the same functional result is administered to a patient suffering from cancer cachexia and/or metastasis associated with end-stage cancer

One aspect of the disclosure provides methods for treating cachexia, the method comprising administering a therapeutically effective amount of an anti-PECAM-1 antibody via systemic administration to a mammal suffering from cachexia. In certain implementations, the mammal is a human. In some implementations, the anti-PECAM-1 antibody is a monoclonal antibody. In some implementations, the mammal is suffering from a metastatic cancer. In certain implementations, the method further comprises administering a second bioactive agent, preferably a compound useful for treating cachexia or an anti-neoplastic agent.

Another aspect of the disclosure provides methods of treating metastasis in a mammal suffering from cachexia, said method comprising administering a therapeutically effective amount of an anti-PECAM-1 antibody via systemic administration to the mammal, whereby metastatic spread or overall tumor burden is decreased. In certain implementations, the mammal is a human. In some implementations, the anti-PECAM-1 antibody is a monoclonal antibody. In certain implementations, the method further comprises administering a second bioactive agent, preferably an anti-neoplastic agent.

The present disclosure provides methods for treating end-stage metastatic cancer and/or tumor-induced cachexia, the method comprising administering a therapeutically effective amount of an anti-PECAM-1 antibody to a mammal suffering from end-stage metastatic cancer and/or tumor-induced cachexia. In certain implementations, the anti-PECAM-1 antibody is human antibody or a humanized antibody.

In some implementations, the method of treating end-stage metastatic cancer by administering a therapeutically effective dose of an anti-PECAM-1 antibody further comprises administering a second bioactive agent. In some implementations, the second bioactive agent is an antineoplastic agent. In one representative implementation, the second bioactive agent is an anti-cancer small molecule. In one representative implementation, the second bioactive agent is an anti-cancer antibody.

In certain implementations, the method of treating cachexia, particularly tumor-induced cachexia, by administering a therapeutically effective dose of an anti-PECAM-1 antibody further comprises administering a second bioactive agent. In some implementations, the second bioactive agent is a compound (other than an anti-PECAM-1 antibody) useful for treating cachexia. In one representative implementation, the second compound useful for treating cachexia is an appetite stimulant.

In certain other aspects of the present disclosure there are provided therapeutic kits comprising in suitable container, a pharmaceutical formulation of an anti-PECAM-1 antibody. In one implementation, the kit comprises a unit dose form of a pharmaceutical composition for treating cachexia, said pharmaceutical composition comprising a therapeutically effective amount of an anti-PECAM-1 antibody suitable for systemic administration to a mammal suffering from cachexia.

The details of one or more implementations are set forth in the accompanying drawings and the description below. Other features, objects, and advantages will be apparent from the description and drawings, and from the claims.

### V. BRIEF DESCRIPTION OF THE FIGURES

FIG. 1 summarizes the percent lung area occupied by B16-F10 melanoma after five doses or six doses of anti-PECAM-1 MAb or isotype matched control mAb.

FIG. 2 summarizes the mean number of B16-F10 ovarian metastases after five doses or six doses of anti-PECAM-1 MAb or isotype matched control mAb.

FIG. 3 illustrates the mean body weight of animals after five doses or six doses of anti-PECAM-1 MAb or isotype matched control mAb.

FIG. 4. Mean tumor lung weights. Values mean±s.e.m. N=10.

### VI. DETAILED DESCRIPTION

As used herein and in the appended claims, the singular forms “a,” “and,” and “the” include plural referents unless the context clearly dictates otherwise. Thus, for example, reference to “an antigen” includes a plurality of such antigens and reference to “the immune cell” includes reference to one or more immune cells known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

“Cachexia” refers to a state of general ill health and malnutrition. It is often associated with and induced by malignant cancer and is characterized by loss of appetite, loss of body mass, especially lean body mass, and muscle wasting. “Anorexia” refers simply to a loss of appetite, whether brought on by medical, physiological or psychological factors. Anorexia is often closely associated with, and generally contributes to, cachexia seen in patients with advanced cancers and other conditions.

The term “therapeutically effective amount” as used herein refers to that amount which provides therapeutic effects for a given condition and administration regimen. In the context of cachexia, therapeutic effects would include, for example, slowing, stopping or reversing weight gain, maintaining or recovering physical function, and/or appetite increase. In the context of end-stage metastasis, therapeutic effects would include function, and/or appetite increase. In the context of end-stage metastasis, therapeutic effects could include arrest of tumor growth and/or tumor shrinkage, increased activity and performance status, weight gain, pain reduction and increased appetite.

As used herein, the term “systemic administration” refers to parenteral (including intramuscular, subcutaneous and intracutaneous), intraluminal, intraarterial, intrathecal and/or intranasal administration or by direct injection into tissue.

The term “naturally-occurring” as used herein as applied to an object refers to the fact that an object can be found in nature. For example, compounds present in a plan, or a polypeptide or polynucleotide sequence that is present in an organism (including viruses) that can be isolated from a source in nature and which has not been intentionally modified by man in the laboratory is naturally-occurring. Generally, the term naturally occurring refers to an object as present in a nonpathological (undiseased) individual, such as would be typical for the species.

The term “agent” is used herein to denote a chemical compound, a mixture of chemical compounds, an array of spatially localized compounds (e.g., a VLSIPS peptide array, polynucleotide array, and/or combinatorial small molecule array), a biological macromolecule, a bacteriophage peptide display library, a bacteriophage antibody (e.g., scFv) display library, a polysome peptide display library, or an extract made from biological materials such as bacteria, plants, fungi, or animal (particularly mammalian) cells or tissues. Agents are evaluated for potential activity as antineoplastics, anti-inflammatories, or apoptosis modulators by inclusion in screening assays described herein. Agents are evaluated for potential activity as specific protein interaction inhibitors (i.e., an agent which selectively inhibits a binding interaction between two predetermined polypeptides but which does not substantially interfere with cell viability) by inclusion in screening assays described herein.

The term “antineoplastic agent” is used herein to refer to agents that have the functional property of inhibiting a development or progression of a neoplasm in a human, particularly a metastasis-prone solid tumor type.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas. The term “antibody” is used in the broadest sense and specifically covers, without limitation, intact monoclonal antibodies, polyclonal antibodies, multispecific antibodies (e.g., bispecific antibodies) formed from at least two intact antibodies, and antibody fragments so long as they exhibit the desired biological activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

PECAM-1 is a 130 kDa cell surface protein of the Ig-like superfamily, with six Ig-like domains in the extracellular domain. It is expressed on certain white blood cells (WBC), platelets and vascular endothelial cells (VEC), and interacts homophilically with itself, or heterophilically with putative ligands to tranduce downstream inhibitory signals via its cytoplasmic domain. PECAM-1 is involved in a number of processes relevant to growth and spread of primary tumors, including angiogenesis, vascular permeability and leukocyte trafficking out of the circulation. In addition, earlier studies have shown that systemic delivery of an anti-PECAM-1 ribozyme suppresses the progression of already established tumor metastases (Kashani-Sabat, et al. 2002. Proc Nat'l Acad Sci USA99:3878-3883).

U.S. Pat. No. 7,572,443, incorporated herein by reference, discloses novel anti-PECAM-1 therapies relating to treating metastatic neoplastic diseases. Platelet endothelial cell adhesion molecule 1 (PECAM-1) is a 130-kDa cell surface protein of the Ig-like superfamily, with six Ig-like domains in the extracellular domain that is expressed on certain white blood cells, platelets, and vascular endothelial cells (VEC). This patent demonstrated that systemic delivery of an anti-PECAM-1therapy suppresses the progression of already established tumor metastasis.

In this disclosure, it is demonstrated that anti-PECAM-1therapies suppress both end-stage metastatic progression and tumor-induced cachexia in patients with advanced cancers. Anti-PECAM-1 therapies produce potent antimetastatic effects specifically against the lethal pre-terminal stage of metastatic progression. These anti-PECAM-1therapies act independently of tumor type as their effects are mediated by binding to vascular endothelial cell (VEC) rather than to tumor cells.

In certain implementations, the methods and compositions are used to treat metastatic tumors and/or cachexia in patients suffering form a metastatic tumor. Examples of metastatic tumors that can be treated or prevented according to a method of the present disclosure include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastric cancer, pancreatic cancer, breast cancer, ovarian cancer, fallopian tube cancer, primary carcinoma of the peritoneum, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, lymphoma, neuroblastoma, and retinoblastoma. In certain implementations, the metastatic tumor is selected from the group consisting of colon carcinoma, breast cancer, lung carcinoma, melanoma and lymphoma. In one implementation, the metastatic tumor is melanoma. In another implementation, the metastatic tumor is breast cancer.

Early stages of metastasis form micrometastatic foci. The well-established metastases turn to organ-destructive growth, and lead to lethal pre-terminal stage. Vascular endothelial cell PECAM regulates this lethal progression of advanced tumor metastasis. Dose-intensive, pre-terminal administration of anti-PECAM-1therapies suppress the treatment-refractory end stage of metastatic progression.

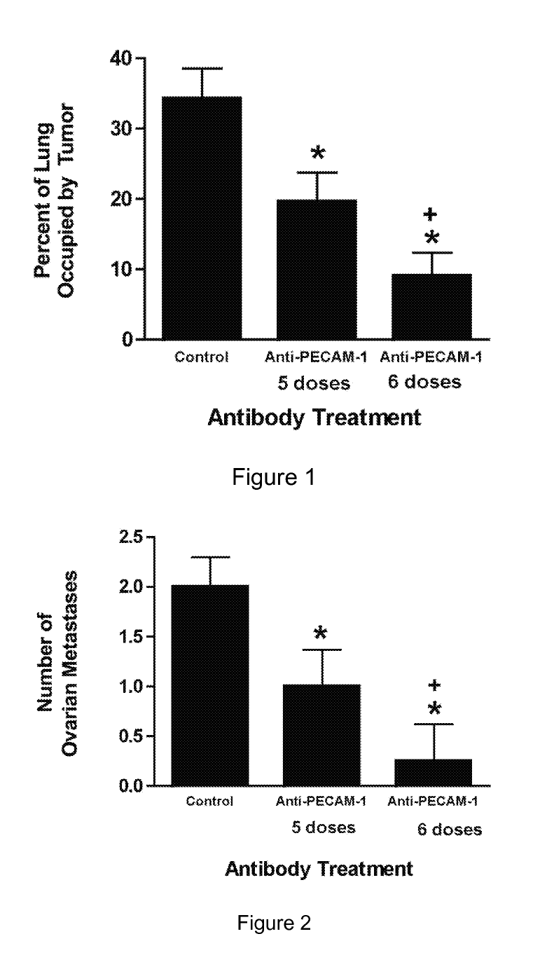

Remarkably, dose-intensive, pre-terminal mAb administration effectively treats the treatment-refractory, end-stages of metastatic progression. Specifically, one pre-terminal dose of anti-PECAM-1 mAb was itself strikingly effective, further reducing lung metastatic burden by an additional 50% against B16-F10 (FIG. 1) and an additional 35% against 4T1 tumors (data not shown) Although many anti-cancer treatments are significantly more toxic in hosts already debilitated from advanced tumor metastases, high doses of anti-PECAM-1 mAb administered during the pre-terminal phase not only appear non-toxic, but also significantly reduce tumor-induced cachexia. Additionally, PECAM-1 knockout mice grow and develop normally (Duncan, et al. 1999. J Immunol 162:3022-3030). Taken together, these results indicate that anti-PECAM-1-mAb therapy is expected to be well tolerated, even in human patients already severely debilitated from advanced metastases.

While pre-clinical metastasis studies often focus on the early stages of metastatic spread, the study of pre-terminal metastatic progression has largely been avoided. In contrast, clinical trials of investigational anti-cancer agents, selected based on early-stage pre-clinical studies, are often tested in patients bearing late-stage metastases, suggesting a mismatch between models studied and patients treated. Anti-PECAM-1 mAb's striking efficacy against pre-terminal metastases, coupled with its inactivity against early-stage metastatic spread suggest that the lethal growth of advanced tumor metastases are, at least in part, controlled by late stage-specific, pro-metastatic drivers. Furthermore, these results indicate that late stage-specific testing of novel anti-tumor agents will reveal some agents active only during this usually treatment-resistant phase of metastatic progression. Such agents may include novel agents previously determined to be ‘ineffective’ when tested solely against early-stage metastatic spread.

This apparent anomaly may in part be due to the largely proliferation-driven nature of advanced metastatic disease. Conversely, the successful establishment of micro-metastatic foci depends upon the abilities of tumor cells to locally invade, intravasate, home to distant organs, extravasate and induce tumor neo-angiogenesis. IV injected, non-tumorigenic, mammary gland cells can colonize and grow within the lung for prolonged periods, further indicating that early metastatic spread occurs by a series of discrete stages. Taken together, our studies indicate that a complex interplay between elements of the tumor microenvironment, paracrine factors and advanced tumor metastases controls the lethal progression of advanced tumor metastases. Selectively targeting PECAM-1 represents a novel, tumor microenvironment-targeted therapeutic that suppresses even lethal, end-stage metastatic progression, up to now a refractory clinical entity.

Accordingly one aspect of the disclosure relates to use of a mammalian antibody or fragment thereof which specifically binds to PECAM-1. The term “specifically binds” or related expressions such as “specific binding”, “binding specifically”, “specific binder” etc. as used herein refer to the ability of the human antibody or fragment thereof to discriminate between PECAM-1, preferably human PECAM-1, and any number of other potential antigens different from PECAM-1 to such an extent that, from a pool of a plurality of different antigens as potential binding partners, only PECAM-1 is bound, or is significantly bound. As used herein, PECAM-1 is “significantly” bound when, from among a pool of a plurality of equally accessible different antigens as potential binding partners, PECAM-1 is bound at least 10-fold, preferably 50-fold, most preferably 100-fold or greater more frequently (in a kinetic sense) than any other antigen different than PECAM-1. Such kinetic measurements can be performed, for example, on a Biacore apparatus.

In one implementation, the anti-PECAM-1 antibody is selected from the group consisting of mAb390 (Millipore), TLD-3A12 (Millipore), P2B1 (Millipore), 2H8 (Millipore), HC1/6 (Millipore), Mec13.3 (Pharmingen, and described in Eur J Cell Biol 1994, 63:247-254), mAb 37 or mAb 62 (described in J Immunol 164: 452-462, 2000). In another implementation, the anti-PECAM-1 antibody is a monoclonal antibody that binds to domain 2 of PECAM-1. One exemplary antibody with an epitope in domain 2 is mAb 390.

In certain implementations, the antibody or fragment thereof is monoclonal. As used herein, the term “monoclonal” is to be understood as having the meaning typically ascribed to it in the art, namely an antibody (or its corresponding fragment) arising from a single clone of an antibody-producing cell such as a B cell, and recognizing a single epitope on the antigen bound. In certain implementations, the monoclonal antibody is a human monoclonal antibody. It is particularly difficult to prepare human antibodies which are monoclonal. In contrast to fusions of murine B cells with immortalized cell lines, fusions of human B cells with immortalized cell lines are not viable. Thus, a human monoclonal antibody is the result of overcoming significant technical hurdles generally acknowledged to exist in the field of antibody technology. The monoclonal nature of the antibody makes it particularly well suited for use as a therapeutic agent, since such antibody will exist as a single, homogeneous molecular species which can be well-characterized and reproducibly made and purified. These factors result in a product whose biological activity can be predicted with a high level of precision, very important if such a molecule is going to gain regulatory approval for therapeutic administration in humans.

When used to treat cancer cachexia in humans, it is important that the monoclonal antibody (or corresponding fragment) be a human antibody (or corresponding fragment). In contemplating an antibody agent intended for therapeutic administration to humans, it is highly advantageous that this antibody is of human origin. Following administration to a human patient, a human antibody or fragment thereof will most probably not elicit a strong immunogenic response by the patient's immune system, i.e. will not be recognized as being a “foreign”, that is non-human protein. This means that no host, i.e. patient antibodies will be generated against the therapeutic antibody which would otherwise block the therapeutic antibody's activity and/or accelerate the therapeutic antibody's elimination from the body of the patient, thus preventing it from exerting its desired therapeutic effect.

The term “human” antibody as used herein is to be understood as meaning that the antibody, or its fragment, comprises (an) amino acid sequence(s) contained in the human germline antibody repertoire. For the purposes of definition herein, an antibody, or its fragment, may therefore be considered human if it consists of such (a) human germline amino acid sequence(s), i.e. if the amino acid sequence(s) of the antibody in question or fragment thereof is (are) identical to (an) expressed human germline amino acid sequence(s). An antibody or fragment thereof may also be regarded as human if it consists of (a) sequence(s) that deviate(s) from its (their) closest human germline sequence(s) by no more than would be expected due to the imprint of somatic hypermutation. Additionally, the antibodies of many non-human mammals, for example rodents such as mice and rats, comprise VH CDR3 amino acid sequences which one may expect to exist in the expressed human antibody repertoire as well. Any such sequence(s) of human or non-human origin which may be expected to exist in the expressed human repertoire would also be considered “human” for the purposes of the present disclosure.

According to a further implementation, the fragment of the human monoclonal antibody may be an scFv, a single domain antibody, an Fv, a VHH antibody, a diabody, a tandem diabody, a Fab, a Fab′ or a F(ab)₂. These formats may generally be divided into two subclasses, namely those which consist of a single polypeptide chain, and those which comprise at least two polypeptide chains. Members of the former subclass include an scFv (comprising one VH region and one VL region joined into a single polypeptide chain via a polypeptide linker); a single domain antibody (comprising a single antibody variable region) such as a VHH antibody (comprising a single VH region). Members of the latter subclass include an Fv (comprising one VH region and one VL region as separate polypeptide chains which are non-covalently associated with one another); a diabody (comprising two non-covalently associated polypeptide chains, each of which comprises two antibody variable regions—normally one VH and one VL per polypeptide chain—the two polypeptide chains being arranged in a head-to-tail conformation so that a bivalent antibody molecule results); a tandem diabody (bispecific single-chain Fv antibodies comprising four covalently linked immunoglobulin variable—VH and VL—regions of two different specificities, forming a homodimer that is twice as large as the diabody described above); a Fab (comprising as one polypeptide chain an entire antibody light chain, itself comprising a VL region and the entire light chain constant region and, as another polypeptide chain, a part of an antibody heavy chain comprising a complete VH region and part of the heavy chain constant region, said two polypeptide chains being intermolecularly connected via an interchain disulfide bond); a Fab′ (as a Fab, above, except with additional reduced disulfide bonds comprised on the antibody heavy chain); and a F(ab)₂ (comprising two Fab′ molecules, each Fab′ molecule being linked to the respective other Fab′ molecule via interchain disulfide bonds). In general, antibody fragments of the type described herein allow great flexibility in tailoring, for example, the pharmacokinetic properties of an antibody desired for therapeutic administration to the particular exigencies at hand. For example, it may be desirable to reduce the size of the antibody administered in order to increase the degree of tissue penetration when treating tissues known to be poorly vascularized (for example, large solid tumors). Under some circumstances, it may also be desirable to increase the rate at which the therapeutic antibody is eliminated from the body, said rate generally being accelerated by decreasing the size of the antibody administered.

According to a further implementation, said monoclonal antibody or fragment thereof may be present in monovalent monospecific; multivalent monospecific, in particular bivalent monospecific; or multivalent multispecific, in particular bivalent bispecific forms. In general, a multivalent monospecific, in particular bivalent monospecific antibody such as a full human IgG as described hereinabove may bring with it the therapeutic advantage that the neutralization effected by such an antibody is potentiated by avidity effects, i.e. binding by the same antibody to multiple molecules of the same antigen, here PECAM-1. Several monovalent monospecific forms of fragments of the antibody have been described above (for example, an scFv, an Fv, a VHH or a single domain antibody). Multivalent multispecific, in particular bivalent bispecific forms of the human monoclonal anti-PECAM-1 antibody may include a full IgG in which one binding arm binds to PECAM-1 while the other binding arm of which binds to another antigen different from PECAM-1. A further multivalent multispecific, in particular bivalent bispecific form may advantageously be a human single chain bispecific antibody, i.e. a recombinant human antibody construct comprising two scFv entities as described above, connected into one contiguous polypeptide chain by a short interposed polypeptide spacer as generally known in the art (see for example WO 99/54440 for an anti-CD19×anti-CD3 bispecific single chain antibody). Here, one scFv portion of the bispecific single chain antibody comprised within the bispecific single chain antibody will specifically bind PECAM-1 as set out above, while the respective other scFv portion of this bispecific single chain antibody will bind another antigen determined to be of therapeutic benefit.

According to a further implementation the monoclonal antibody or fragment thereof may be derivatized, for example with an organic polymer, for example with one or more molecules of polyethylene glycol (“PEG”) and/or polyvinyl pyrrolidone (“PVP”). As is known in the art, such derivatization can be advantageous in modulating the pharmacodynamic properties of antibodies or fragments thereof. Especially preferred are PEG molecules derivatized as PEG-maleimide, enabling conjugation with the antibody or fragment thereof in a site-specific manner via the sulfhydryl group of a cysteine amino acid. Of these, especially preferred are 20 kD and/or 40 kD PEG-maleimide, in either branched or straight-chain form. It may be especially advantageous to increase the effective molecular weight of smaller human anti-PECAM-1 antibody fragments such as scFv fragments by coupling the latter to one or more molecules of PEG, especially PEG-maleimide.

In accordance with this disclosure, the term “pharmaceutical composition” relates to a composition for administration to a patient, preferably a human patient. In a preferred implementation, the pharmaceutical composition comprises a composition for parenteral, transdermal, intraluminal, intraarterial, intrathecal and/or intranasal administration or by direct injection into tissue. It is in particular envisaged that said pharmaceutical composition is administered to a patient via infusion or injection. Administration of the suitable compositions may be effected by different ways, e.g., by intravenous, intraperitoneal, subcutaneous, intramuscular, topical or intradermal administration. The pharmaceutical composition may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, liposomes, etc. Compositions comprising such carriers can be formulated by well known conventional methods.

These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

The disclosure further provides a method of treating end-stage metastatic cancer and/or tumor-induced cachexia in a patient by administering one or more doses of an anti-PECAM-1 antibody, wherein each dose is between about 0.1 to about 30 mg/kg, or about 0.5 to about 20 mg/kg, or about 1.0 to about 15 mg/kg, or about 2.0 to about 12 mg/kg, or about 3 to about 10 mg/kg. In some implementations, doses may be administered at an interval of about once each day or longer, or once every other day or longer, or once every three days or longer, or once per week or longer, or once each 2 weeks or longer, or once every month or longer. The anti-PECAM-1 antibody may be used in the preparation of a medicament for administration using any of the dosing and timing regimens described herein. Optionally, the anti-PECAM-1 antibody is presented in a container, such as a single dose or multidose vial, containing a dose of anti-PECAM-1 antibody for administration (e.g., about 500 to about 1500 mg of anti-PECAM-1 antibody). In one exemplary implementation, a vial may contain about 800 mg or 850 mg of anti-PECAM-1 antibody and would be suitable for administering a single dose of about 10 mg/kg. In other implementations, a vial may contain about 1600 mg or 1700 mg; or about 2400 mg or 2500 mg or 2550 mg of anti-PECAM-1 antibody.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the pharmaceutical composition might comprise proteinaceous carriers, like, e.g., serum albumin or immunoglobulin, preferably of human origin.

It is also possible and contemplated that the anti-PECAM-1 antibodies according to several implementations are used in combination with other drugs or agents, particularly in the treatment of cachexia. These other drugs and agents may include agents that induce weight gain, including corticosteroids and progestational agents, appetite stimulants, anti-inflammatories, and/or drugs targeting protein catabolism. In one implementation, anti-PECAM-1 antibodies are used in combination with a therapeutically effective amount of a second weight gain pharmaceutical agent.

According to another implementation, methods for the treatment of cachexia are provided, the method including the step of administering to the patient having or at risk of having cachexia a therapeutically effective amount of an anti-PECAM-1 antibody in combination with a therapeutically effective amount of another compound that is useful in the treatment of cachexia. An implementation also provides pharmaceutical compositions that comprise 1) an anti-PECAM-1 antibody and 2) a second compound useful for the treatment of cachexia.

The second compounds useful for the treatment of cachexia are preferably selected from but not limited to the group consisting of ADP-ribose-polymerase inhibitors, ADP-ribose-transferase inhibitors, NADase inhibitors, nicotinamide benzamide, theophylline, thymine and analogs thereof; omega-3 fatty acids such as alpha-linolenic acid, stearidonic acid, eicosapentaenoic acid (EPA), docosapentaenoic acid, docosahexaenoic acid or mixtures thereof; branched-chain amino acids valine, leucine, isoleucine or mixtures thereof, with or without reduced levels of tryptophan and 5-hydroxytryptophan; antioxidants selected from the group comprising beta-carotene, vitamin C, vitamin E, selenium, or mixtures thereof; L-glutamine, vitamin A, vitamin C, vitamin E, and selenium; Azaftig; quinine derivatives including 3,5,6-trimethyl-2-(3-pyridyl)methyl-1,4-benzoquinone hydrochloride; interleukin 2; benzaldehyde; 4,6-O-benzylidene-D-glucose; friedelan-3-one; hydrazine sulfate; medroxyprogesterone acetate; beta 2-adrenoceptor agonists; corticosteroids such as dexamethasone; megestrol acetate; dronabinol; thalidomide; fluoxymesterone; pentoxifylline; cyproheptadine; metoclopramide; somatotropin, total parenteral nutrition; melanocortin-4 receptor antagonists; ghrelin receptor agonists. cannabinoids, TNF-α antagonists, adrenoreceptor agonists, adrenoreceptor antagonists, and/or modulators of muscle protein synthesis or degradation. In one representative implementation, the second compound useful for treating cachexia is an appetite stimulant. In another representative implementation, the second compound useful for treating cachexia is megestrol acetate. In yet another representative implementation, the second compound useful for treating cachexia is a cannabinoid derivative. In yet another representative implementation, the therapeutic agent targets inflammatory cytokines. In one representative implementation, the second compound is a TNF-α inhibitor. In yet another representative implementation, the second compound targets protein catabolism. In one representative implementation, the second compound is a β-adrenoreceptor agonist. In yet another representative implementation, the second compound is an anabolic steroid.

It is envisaged that the pharmaceutical composition might comprise, in addition to the anti-PECAM-1 antibody or fragment thereof, a second bioactive agent. Other suitable bioactive agents include, for example, antineoplastic agents, such as platinum compounds (e.g., spiroplatin, cisplatin, and carboplatin), methotrexate, adriamycin, taxol, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), interferon α-2a, interferon α-2b, teniposide (VM-26), vinblastine sulfate (VLB), vincristine sulfate, bleomycin, bleomycin sulfate, methotrexate, adriamycin, and arabinosyl; blood products such as parenteral iron, hemin, hematoporphyrins and their derivatives; hormones and steroids such as growth hormone, melanocyte stimulating hormone, estradiol, beclomethasone dipropionate, betamethasone, betamethasone acetate and betamethasone sodium phosphate, vetamethasone disodium phosphate, vetamethasone sodium phosphate, cortisone acetate, dexamethasone, dexamethasone acetate, dexamethasone sodium phosphate, flunsolide, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide and fludrocortisone acetate; vitamins such as cyanocobalamin neinoic acid, retinoids and derivatives such as retinol palmitate, and α-tocopherol; anti-allergic agents such as amelexanox; metabolic potentiators such as glutathione; anti-inflammatories such as diffinisal, ibuprofen, indomethacin, meclofenamate, mefenamic acid, naproxen, oxyphenbutazone, phenylbutazone, piroxicam, sulindac, tolmetin, aspirin and salicylates; narcotics such as paregoric; opiates such as codeine, heroin, methadone, morphine and opium; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; and radioactive particles or ions such as strontium, iodide rhenium and yttrium. In certain preferred implementations, the bioactive agent is an antineoplastic agent. In some implementations, the second bioactive agent is an antineoplastic agent. In one representative implementation, the second bioactive agent is an anti-cancer small molecule. In one representative implementation, the second bioactive agent is an anti-cancer antibody.

In a further aspect, provided herein are kits comprising one or more unit dose forms as described herein. In some implementations, the kit comprises one or more of packaging and instructions for use to treat one or more diseases or conditions. In some implementations, the kit comprises a diluent which is not in physical contact with the active agent or pharmaceutical composition. In some implementations, the kit comprises any of one or more unit dose forms described herein in one or more sealed vessels. In some implementations, the kit comprises any of one or more sterile unit dose forms.

In some implementations, the kit comprises a container for the pharmaceutical formulation of the present disclosure. Suitable containers include, for example, a bottle, a vial, a box, or a combination thereof. Optionally, the kit also contains directions for properly administering the formulations. The kits can also be designed in a manner such that they are tamper resistant or designed to indicate if tampering has occurred. Optionally, the kit can contain the anti-PECAM-1 antibody in combination with other pharmaceutical compositions. In some implementations, the anti-PECAM-containing pharmaceutical composition is an individual dosage unit.

In certain other aspects, the therapeutic kits comprise, in a suitable container, a pharmaceutical formulation of an anti-PECAM-1 antibody. Such a kit may further comprise a pharmaceutical formulation of a second bioactive agent compound, such as a naturally-occurring compound, polypeptide, polynucleotide encoding a therapeutic polypeptide, or therapeutic agent. Such kits may comprise therapeutic agents, instructions for administration of an anti-PECAM-1 antibody to a human patient diagnosed with a neoplasm, particularly a lung, ovarian, breast neoplasm or in a variation a melanoma, via systemic delivery.

Optionally associated with the container(s) in the kits can be a notice or printed instructions. Such printed instructions can be in a form prescribed by a governmental agency regulating the manufacture, use, or sale of pharmaceuticals or biological products, which notice reflects approval by the agency of the manufacture, use, or sale for human administration to treat a condition that could be treated by sympathomimetic drug therapy. In some implementations, the kit further comprises printed matter, which, e.g., provides information on the use of the pharmaceutical composition to treat a condition or disease or a pre-recorded media device which, e.g., provides information on the use of the pharmaceutical composition to treat a condition or disease, or a planner.

The kit can also include a container for storing other components of the kit. The container can be, for example, a bag, box, envelope or any other container that would be suitable for use. Preferably, the container is large enough to accommodate each component and/or any administrative devices that may be accompany the formulations described herein.

The publications discussed above and throughout the text are provided solely for their disclosure prior to the filing date of the present application. Nothing herein is to be construed as an admission that the inventors are not entitled to antedate such disclosure by virtue of prior disclosure.

The working examples below are provided to illustrate, not limit, the disclosure. Various parameters of the scientific methods employed in these examples are described in detail below and provide guidance for practicing the disclosure in general.

### VII. EXAMPLES

### A. Effectiveness of Anti-PECAM-1-mAb Therapy Against Both End-Stage Metastatic Progression and Tumor Induced Cachexia in Tumor-Bearing Mice

Anti-PECAM-1 mAb390 was tested specifically against the pre-terminal stage of metastatic progression. Anti-PECAM-1 mAb390 is a bioactive anti-murine PECAM-1 mAb, which specifically binds to an epitope within a 14 amino acid sequence of the second Ig-like domain of mouse PECAM-1. mAb390 binds to murine VEC, but does not bind to murine or human tumors (Zhou, et al. 1999. Angiogenesis 3:181-188). Furthermore, it does not directly inhibit tumor cell proliferation; alter tumor cell adhesion to VEC or to platelets or the transendothelial migration of tumor cells (data not shown). Anti-PECAM-1 mAb390 has been shown to specifically inhibit PECAM-1-dependent activities, and functions similarly to a number of other anti-PECAM-1 antibodies (which bind to epitopes distinct from that of mAb390), in a number of both in vitro, and in vivo assays.

The effects of administering one additional dose to mice already severely ill from extensive B16-F10 tumor metastases was assessed. C57BL/6 mice received 25,000 B16-F10 cells injected intravenously on day 0. IV-injected B16-F10 cells yield highly reproducible numbers of tumor metastases in the lung, as well as in extrapulmonary organs, including the ovaries. Each mouse (n=10) received (a) one i.v. injection of 200 μg anti-PECAM-1 on day 7 and subsequently received one dose every other day through day 15 (five mAb doses), (b) one i.v. injection of 200 μg anti-PECAM-1 on day 7 and subsequently received one dose every other day through day 15, plus one dose on day 18 (six mAb doses), or (c) one i.v. injection of isotype control mAb on day 7 and subsequently received one dose every other day through day 15 plus one dose on day 18 (six control mAb doses). Mice were killed when multiple control mice became moribund.

Images of all histologic sections in the anti-PECAM-1-mAb and control groups were captured using a Nikon Digital Sight D5-U1 camera and Nikon 80i microscope. Surface areas occupied by total lung and individual tumor masses within the lung were outlined using the area function of NIS-Elements Software (Nikon Instruments), and percentage of lung occupied by tumor was calculated. The pre-terminal, anti-PECAM-1-mAb dose (sixth dose) itself significantly reduced the percentage of lung occupied by tumor metastases: control-group: 34.3+4.2%, standard-5-dose mAb390 group: 19.6+4.2% (p<0.05 versus control) and extended-6-dose group: 9.0+3.3% (p<0.0001 versus control, p<0.05 versus 5-doses) (FIG. 1). There were significant differences among the three treatments (P=0.001 by Kruskal-Wallis rank test). Also, all pairwise comparisons were significantly different: five doses vs. none, P=0.02; six doses vs. none, P=0.002; and five doses vs. six doses, P=0.02; all pairwise tests were by Mann-Whitney rank test. Administration of the pre-terminal dose also reduced (p<0.001) ovarian metastases (FIG. 2), indicating that anti-PECAM-1-mAb therapy is systemically active. Potential statistical significance of differences for were assessed using pair wise two-sided Student's t-tests: *p<0.05 6 doses vs control; and +p<0.05 6 doses vs 5 doses.

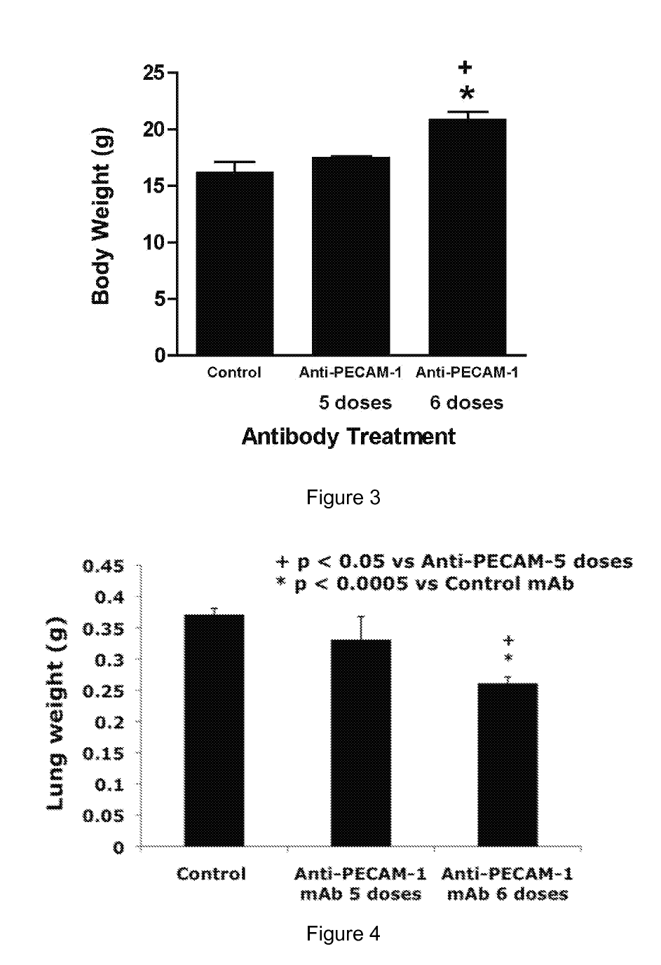

Additionally, administration of the pre-terminal dose increased total body weight, determined at the time of sacrifice (p<0.0005), when compared to either mice not receiving the pre-terminal dose (sixth dose) or control groups (FIG. 3). There were significant differences among the three treatments (P=0.002 by Kruska-Wallis rank test). Also, six doses vs. none (P=0.003) and five doses vs. six doses (P=0.004) were significantly different. All pairwise tests were by Mann-Whitney rank test. Concurrently, lung tumor weights were significantly reduced in the group receiving the pre-terminal dose (FIG. 4), showing that the pre-terminal dose preserved normal body weight, while concurrently reducing the overall tumor burden and total tumor weight. Taken together, these results indicate that targeting PECAM-1 reduces tumor-induced cachexia.

BALB/c mice received 25,000 4T1 cells i.v. and then received five doses (last dose on day 15) or six doses (last dose on day 18) of anti-PECAM-1 or isotype control mAb. Mice were killed when multiple control mice became moribund. Values represent mean±SEM (n=10). In C and E, potential statistical significance of differences was assessed using pairwise two-sided Student's t tests. *P<0.05 vs. control; +P<0.05 vs. five doses.

A number of implementations have been described. Nevertheless, it will be understood that various modifications may be made without departing from the spirit and scope of the description. Accordingly, other implementations are within the scope of the following claims.

What is claimed is:

1. A method for treating cachexia, the method comprising administering a therapeutically effective amount of an anti-PECAM-1 antibody via systemic administration to a mammal suffering from cachexia.

2. The method of claim 1, wherein the mammal is a human.

3. The method of claim 1, wherein the antibody is a monoclonal antibody.

4. The method of claim 1, wherein the mammal is suffering from a metastatic cancer.

5. The method of claim 1, wherein the therapeutically effective amount is between about 0.1 mg/kg and about 30 mg/kg.

6. The method of claim 1, wherein the therapeutically effective amount is between about 1.0 and about 15 mg/kg.

7. The method of claim 1, wherein the method further comprising administering a second bioactive agent.

8. The method of claim 7, wherein the second bioactive agent is a compound useful for treating cachexia.

9. The method of claim 8, wherein the compound useful for treating cachexia is selected from the group consisting of ADP-ribose-polymerase inhibitors, ADP-ribose-transferase inhibitors, NADase inhibitors, nicotinamide benzamide, theophylline, thymine and analogs thereof; omega-3 fatty acids such as alpha-linolenic acid, stearidonic acid, eicosapentaenoic acid (EPA), docosapentaenoic acid, docosahexaenoic acid or mixtures thereof; branched-chain amino acids valine, leucine, isoleucine or mixtures thereof, with or without reduced levels of tryptophan and 5-hydroxytryptophan; antioxidants selected from the group comprising beta-carotene, vitamin C, vitamin E, selenium, or mixtures thereof; L-glutamine, vitamin A, vitamin C, vitamin E, and selenium; Azaftig; quinine derivatives including 3,5,6-trimethyl-2-(3-pyridyl)methyl-1,4-benzoquinone hydrochloride; interleukin 2; benzaldehyde; 4,6-O-benzylidene-D-glucose; friedelan-3-one; hydrazine sulfate; medroxyprogesterone acetate; beta 2-adrenoceptor agonists; corticosteroids such as dexamethasone; megestrol acetate; dronabinol; thalidomide; fluoxymesterone; pentoxifylline; cyproheptadine; metoclopramide; somatotropin, total parenteral nutrition; melanocortin-4 receptor antagonists; ghrelin receptor agonists; cannabinoids; TNF-α antagonists; adrenoreceptor agonists; adrenoreceptor antagonists; and modulators of muscle protein synthesis or degradation.

10. A method of treating metastasis in a mammal suffering from cachexia, said method comprising administering a therapeutically effective amount of an anti-PECAM-1 antibody via systemic administration to the mammal, whereby metastatic spread or overall tumor burden is decreased.

11. The method of claim 10, wherein the mammal is a human.

12. The method of claim 10, wherein the anti-PECAM-1 antibody is a monoclonal antibody.

13. The method of claim 10, wherein the therapeutically effective amount is between about 0.1 mg/kg and about 30 mg/kg.

14. The method of claim 10, wherein the therapeutically effective amount is between about 1.0 mg/kg and about 15 mg/kg.

15. The method of claim 10, wherein the method further comprising administering a second bioactive agent.

16. The method of claim 15, wherein the bioactive agent an antineoplastic agent.

17. The method of claim 16, wherein the antineoplastic agent is selected from the group consisting of platinum compounds, methotrexate, adriamycin, taxol, mitomycin, ansamitocin, bleomycin, cytosine arabinoside, arabinosyl adenine, mercaptopolylysine, vincristine, busulfan, chlorambucil, melphalan, mercaptopurine, mitotane, procarbazine hydrochloride dactinomycin, daunorubicin hydrochloride, doxorubicin hydrochloride, mitomycin, plicamycin, aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide, interferon α-2a, interferon α-2b, teniposide (VM-26), vinblastine sulfate, vincristine sulfate, bleomycin, bleomycin sulfate, methotrexate, adriamycin, and arabinosyl.

18. A kit comprising a unit dose form of a pharmaceutical composition for treating cachexia, said pharmaceutical composition comprising a therapeutically effective amount of an anti-PECAM-1 antibody suitable for systemic administration to a mammal suffering from cachexia.

19. The kit of claim 18, wherein the unit dose form comprises about 1.0 to about 15 mg/kg of an anti-PECAM-1 antibody for systemic administration to the mammal.
